SEQUENCE LISTING (11 sequences)

SEQ ID NO: 1 - DNA, 47 bp, Artificial Sequence
  Description of Artificial Sequencehir_insf1
atccctgaggaataccttcagcgatttgttaaccaacacttgtgtgg

SEQ ID NO: 2 - protein, 15 aa, Artificial Sequence
  Description of Artificial Sequenceprotein hir_insf1
IPEEYLQRFVNQHLC

SEQ ID NO: 3 - DNA, 47 bp, Artificial Sequence
  Description of Artificial Sequence hir_insrev1
cctcacaagtgttggttaacaaatcgctgaaggtattcctcagggat

SEQ ID NO: 4 - DNA, 46 bp, Artificial Sequence
  Description of Artificial Sequence hirf1
tttttttggatcctttggataaaagacttacgtatactgactgcac

SEQ ID NO: 5 - protein, 6 aa, Artificial Sequence
  Description of Artificial Sequence protein hirf1
LTYTDC

SEQ ID NO: 6 - DNA, 24 bp, Artificial Sequence
  Description of Artificial Sequence insnco1rev
ttttttccatgggtcgactatcag

SEQ ID NO: 7 - DNA, 59 bp, Artificial Sequence
  Description of Artificial Sequence Hir_insf
atccctgaggaataccttcagggaaattcggcacgatttgttaaccaacacttgtgtgg

SEQ ID NO: 8 - protein, 12 aa, Artificial Sequence
  Description of Artificial Sequence protein Hir_insf
GNSARFVNQHLC

SEQ ID NO: 9 - DNA, 59 bp, Artificial Sequence
  Description of Artificial Sequence Hir_insrev
ccacacaagtgttggttaacaaatcgtgccgaatttccctgaaggtattcctcagggat

SEQ ID NO: 10 - DNA, 34 bp, Artificial Sequence
  Description of Artificial Sequence pichia_H_lf1
tttttttctcgagaaaagacttacgtatactgac

SEQ ID NO: 11 - DNA, 41 bp, Artificial Sequence
  Description of Artificial Sequence pichia_H_Irev2
ttttttggcgccgaattcactattagttacagtagttttcc

(19) United States
(12) Patent Application Publication
(10) Pub. No.: US 2003/0176673 A1
(43) Pub. Date: Sep. 18, 2003

(54) Nucleic acids, proteins, and processes thereof such as use of fusion proteins whose N-terminal part is a hirudin derivative for the production of recombinant proteins via secretion by yeasts

(76) Inventor: Paul Habermann, Eppstein (DE)

Correspondence Address: Finnegan, Henderson, Farabow,, Garrett & Dunner, Franklin Square Bldg., Suite 700, 1300 I Street, N.W., Washington, DC, 20005-3315, US

(21) Appl. No.: 10/076,632

(22) Filed: Feb. 19, 2002

Related U.S. Application Data
(60) Provisional application No. 60/270,591, filed on Feb. 23, 2001.

(30) Foreign Application Priority Data
Feb. 20, 2001 (DE) 10108 211.8

(51) Int. Cl. C07H 21/02; C07H 21/04

(52) U.S. Cl. 536/23.1

(57) ABSTRACT

Nucleic acid sequence including: Pₓ—Sₓ—Bₙ—(ZR)-Hir(AsₘR)-protein(Y)-T. Pₓ is a promoter sequence. Sₓ is a nucleic acid encoding a signal sequence or leader sequence. Bₙ is 1-15 codons, when n is an integer from 1 to 15, or a chemical bond, when n=0. Z is a codon for lysine or arginine. R is an arginine codon or a chemical bond. Hir is a nucleic acid sequence coding for hirudin or hirudin derivative which is at least 40% homologous to a natural hirudin isoform. Asₘ is a chemical bond, when m=0, or 1-10 codons, when m is an integer from 1 to 10. Protein(Y) is a nucleic acid sequence encoding a protein that is produced in and secreted by yeast. T is an untranslated expression-enhancing nucleic acid sequence. Proteins thereof, plasmids thereof, multicopy vectors thereof, host cells thereof, and processes thereof.

### CROSS-REFERENCE TO RELATED APPLICATIONS

The present application claims the priority under 35 U.S.C. §119(e) of U.S. Provisional Application No. 60/270,591, filed Feb. 23, 2001, the disclosure of which is expressly incorporated by reference herein in its entirety. The present application also claims the priority under 35 U.S.C. §119 of German Application No. 101 08 211.8, filed Feb. 20, 2001, the disclosure of which is expressly incorporated by reference herein in its entirety.

### DESCRIPTION OF THE INVENTION

The development of optimized processes for producing pharmaceuticals on the basis of recombinant proteins is a task which typically has at least two considerations. First, a process ought to be as cost-effective as possible. Second, the product ought to be of the highest purity. In this connection, the choice of expression system determines the course of the particular production process, and the development of novel techniques in protein chemistry and the wide variety of biochemical possibilities and new combinations of known techniques make improvements of existing processes possible. The expression of relevant proteins of this kind in yeasts is widely used.

The production of proteins such as insulin, GM-CSF (Leukine®) and hirudin (Refludan®) is an example of the successful development of genetic engineering processes which are based on the synthesis of the particular protein or precursors thereof in yeast. Generally, yeasts can directly synthesize hirudins with good yields, which are on the gram scale, when using Hansenula polymorpha (Weydemann et al., Appl. Microbiol Biotechnol. 44:377-385, 1995) or Pichia pastoris (Rosenfeld et al., Protein Expr. Purif: 4, 476-82, 1996).

Surprisingly, we have found that fusion proteins containing hirudin or hirudin derivatives at the N terminus can be exported from yeasts with good yields similar to those of hirudin itself. Yields are based on molarity. This means that a host/vector system producing yields of 100 mg of native hirudin per liter can produce approximately 180 mg fusion protein per liter, which is made of hirudin and, for example, mini-proinsulin, which is described in EP-A 0 347 781. Surprisingly, hirudin is biologically active and mini-proinsulin is present in the correctly folded three-dimensional form. If the two proteins are fused via a linker of amino acids which are specifically recognized by endoproteases which efficiently cleave the fusion protein at no other position, then the protein of interest can be cleaved off directly and in active form. In the case of insulin production, the linker between hirudin and mini-proinsulin may contain arginine at the carboxy-terminal end. In simultaneous processing it is then possible by conversion with trypsin to cleave off the fusion part and convert proinsulin to mono-Arg insulin.

The invention thus may relate to a DNA-molecule of the form:

Pₓ—Sₓ—Bₙ—(ZR)-Hir (AsₘR)-protein(Y)-T,

with the expression cassette coding for hirudin or a hirudin derivative which forms a fusion protein with a protein encoded by protein(Y) via a peptide encoded by AsₘR, where

Pₓ is a promoter DNA sequence which allows optimal yields of the protein of interest to become achievable;

Sₓ is any DNA encoding a signal sequence or leader sequence which allows optimal yields;

Bₙ is 1-15 amino acid codons, when n is an integer from 1 to 15, or a chemical bond, when n=0;

Z is a codon of an amino acid selected from Lys and Arg;

R is an Arg codon;

Asₘ is a chemical bond, when m=0, or m amino acid codons, when m is an integer from 1 to 10;

Hir is a DNA sequence coding for hirudin or a hirudin derivative which is at least 40% homologous to a natural hirudin isoform, such that 40% of the total amount of the 65 amino acids known from lepirudin should be found within the variant. Hir may be at least about 60%, or at least about 80%, homologous to a natural hirudin isoform; protein Y is a DNA sequence encoding any protein which can be produced in and secreted by yeast;

T is an untranslated DNA sequence which is advantageous to expression.

Preferred proteins encoded by protein(Y) are polypeptides such as mini-proinsulin derivatives, interleukins or lymphokines or interferons. Mini-proinsulin is a insulin with a shortened C-chain. A mini-proinsulin derivative is at least 60% homologous to a mini-proinsulin. The term “mini-proinsulin derivative” denotes sequences which are at least 60% homologous to a sequence of a naturally occurring proinsulin. It is understood that the term insulin defines a polypeptide composed out of a B- and A-chain. A mini-proinsulin derivative may be at least about 75%, or at least about 90%, homologous to a mini-proinsulin.

The above % homologies are calculated by the Compare Program, which is available from the Wisconsin Package distributed by the Genetics Computer Group; 575 Science Drive; Madison, Wis. The homology does not cover the C-peptide.

The expression cassette may be introduced into yeasts. Said expression cassette may have one or more copies stably integrated into the particular yeast genome or may be present extrachromosomally on a multicopy vector or on a minichromosomal element.

Another aspect of the invention is a fusion protein encoded by any of the above-mentioned DNA molecules.

Further aspects of the invention include a multicopy vector or a plasmid comprising the above-mentioned DNA-molecule.

An additional aspect of the invention is a host cell comprising the above-mentioned DNA-molecule, or the above-mentioned multicopy vector or the above-mentioned plasmid, as a part of its chromosome, as a part of a mini-chromosome, or extra-chromosomally, wherein preferentially said host cell is a yeast, in particular selected from Saccharomyces cerevisiae, Kluyveromyces lactis, Hansenula polymorpha, and Pichia pastoris.

Another aspect of the invention is a process of fermentative production of the above-mentioned fusion protein, in which

(a) the above-mentioned DNA-molecule, the above-mentioned multicopy vector, or the above-mentioned plasmid is expressed in an above-mentioned host cell, and

(b) the expressed fusion protein is isolated from the supernatant of the cell culture.

For instance, after completion of fermentation, the pH may be adjusted to about 2.5-3.5 in order to precipitate non-desired proteins and the expressed fusion protein is isolated from the supernatant of the precipitation.

Another aspect of the invention is the above mentioned process, in which process after separating the fermentation supernatant from the host cells, the host cells are repeatedly cultured in fresh medium, and the released fusion protein is isolated from each supernatant obtained during cultivation.

Another aspect of the invention is the above mentioned process, wherein a process step for concentrating the expressed protein in the supernatant after precipitation is at least one of microfiltration, hydrophobic interaction chromatography, and ion exchange chromatography.

An additional aspect of the invention is a process for preparing insulin, in which

(a) the above-mentioned fusion protein is expressed and isolated according to the above-mentioned process;

(b) the fusion protein is treated with trypsin and carboxypeptidase B; and

(c) insulin is isolated from the reaction mixture of step (b).

In one aspect, the present invention is directed to a nucleic acid sequence comprising: Pₓ—Sₓ—Bₙ—(ZR)-Hir(AsₘR)-protein(Y)-T. Pₓ is a promoter sequence. Sₓ is a nucleic acid encoding a signal sequence or leader sequence. Bₙ is 1-15 codons, when n is an integer from 1 to 15, or a chemical bond, when n=0. Z is a codon for lysine or arginine. R is an arginine codon or a chemical bond. Hir is a nucleic acid sequence coding for hirudin or hirudin derivative which is at least 40% homologous to a natural hirudin isoform. Asₘ is a chemical bond, when m=0, or 1-10 codons, when m is an integer from 1 to 10. Protein(Y) is a nucleic acid sequence encoding a protein that is produced in and secreted by yeast. T is an untranslated expression-enhancing nucleic acid sequence.

Protein(Y) may encode for mini-proinsulin or a derivative thereof. Protein(Y) may also encode for interleukin, lymphokine, or interferon.

In another aspect, the present invention is directed to a fusion protein encoded by the nucleic acid of the invention.

In still another aspect, the present invention is directed to a multicopy vector comprising the nucleic acid of the invention.

In yet another aspect, the present invention is directed to a plasmid comprising the nucleic acid of the invention.

In a further aspect, the present invention is directed to a host cell comprising the nucleic acid of the invention, as part of the host cell chromosome, as part of a mini-chromosome, or extra-chromosomally.

The host cell may be a yeast which may be selected from Saccharomyces cerevisiae, Kluyveromyces lactis, Hansenula polymorpha, and Pichia pastoris.

In still another aspect, the present invention is directed to a host cell comprising the multicopy vector of the invention.

In another aspect, the present invention is directed to a host cell comprising the plasmid of the invention.

In yet another aspect, the present invention is directed to a process of fermentative production of fusion protein, comprising: expressing the nucleic acid of the host cell of the invention to form the fusion protein in a fermentation supernatant of a cell culture; and isolating the fusion protein from the fermentation supernatant of the cell culture.

The isolating of the fusion protein may comprise adjusting the pH of the fermentation supernatant to about 2.5 to 3.5 to precipitate non-desired proteins and to form a precipitation supernatant, and isolating the fusion protein from the precipitation supernatant.

The process may further comprise separating the fermentation supernatant from the host cell, and after separating the fermentation supernatant from the host cell, the host cell may be repeatedly cultured in fresh medium to form additional supernatant from each culture, and fusion protein may be isolated from each additional supernatant.

The isolation of the fusion protein may comprise precipitating the fusion protein from the fermentation supernatant, and the method may further comprise removing the protein encoded by protein(Y) from the fusion protein, and concentrating the protein encoded by protein(Y) by microfiltration, hydrophobic interaction chromatography, and/or ion exchange chromatography.

In another aspect, the present invention is directed to a process for preparing insulin, comprising: expressing and isolating a fusion protein by one of the above processes; releasing insulin into a reaction mixture by treating the fusion protein with trypsin and carboxypeptidase B; and isolating the insulin from the reaction mixture.

The expression system described below serves as an example. In order to introduce the expression cassette into said selected system, the appropriate recombinant DNA constructions must be made depending on the type of host system selected. Accordingly, industrial fermentation can be optimized in relation to the selected host/vector system.

Leeches of the type Hirudo have developed, for example, various isoforms of the thrombin inhibitor hirudin. Hirudin has been optimized for pharmaceutical requirements by artificial variation of the molecule, for example exchange of the N-terminal amino acid (e.g., EP-A 0 324 712). The invention includes the use of hirudin and hirudin variants. Particular aspects of the invention use one of the natural hirudin isoforms (the natural isoforms are together denoted “hirudin”). A natural isoform is, for example, Val-Val-hirudin or Ile-Thr-hirudin. Other aspects of the invention use a variant of a natural hirudin isoform. A variant is derived from a natural hirudin isoform, but contains, for example, additional amino acids and/or amino acid deletions and/or amino acid exchanges compared with the natural isoform. A hirudin variant may contain alternating peptide segments of natural hirudin isoforms and new amino acids. Hirudin variants are known and are described, for example, in DE 3 430 556. Hirudin variants are commercially available in the form of proteins (Calbiochem Biochemicals, Cat. no. 377-853, -950-960).

Frequently, fusion proteins containing hirudin show surprisingly good solubility in acidic medium, and this leads to distinct advantages regarding the chemical workup of the protein. First, the many components of the supernatant are precipitated under said conditions and, second, most peptidases or proteases are inactive. Thus, acidifying the fermentation broth at the end of the operation makes it possible to directly separate unwanted supernatant proteins together with the host cells from the fusion protein and, in a further step, to concentrate said fusion protein. This is likewise a subject of the invention.

At the end of the fermentation, the folding process may not yet be 100% complete. The addition of mercaptan or, for example, cysteine hydrochloride can complete the process. This is likewise a subject of the invention.

The examples below describe the invention in more detail, without being restrictive.

### EXAMPLE 1

### Construction of an Expression Cassette Encoding a Fusion Protein Made of Leu-Hirudin (Refludan®)-Arg-Mini-Proinsulin

Starting materials were the plasmids pK152 (PCT/EP00/08537, which is incorporated by reference herein in its entirety), pSW3 (EP-A 0 347 781, which is incorporated by reference herein in its entirety) and the recombinant yeast plasmid derivative coding for bovine interleukin 2, which is pαADH2 plus the cDNA for IL2 (Price et al., Gene 55, 1987, which is incorporated by reference herein in its entirety). The yeast plasmid was distinguished by carrying the α factor leader sequence under the control of the yeast ADH2 promoter. This sequence was followed by the bovine interleukin 2 cDNA sequence which was connected via a KpnI restriction enzyme recognition site and which contained, an NcoI restriction enzyme recognition site in the untranslated 3′ end which was unique in the vector. Thus, the cDNA sequence was readily removable from the plasmid via KpnI/NcoI cleavage. Since good expression yields were reported, it was assumed that the remaining 3′ interleukin 2 sequence (as a terminator sequence) had a stabilizing effect on the mRNA and thus need not be replaced by a yeast specific terminator sequence. Plasmid pK152 carried the DNA sequence coding for Leu-hirudin (Refludan®) and plasmid pSW3 carried the DNA sequence for mini-proinsulin. The gene sequence encoding hirudin-Lys Arg-mini-proinsulin was first prepared by means of PCR technology. For this purpose, 4 primers were prepared with the aid of the Expedite™ DNA synthesis system:

i. hir_insf1 (SEQ ID NO: 1, Encoded Protein Segment: SEQ ID NO: 2)

I P E E Y L Q Arg F V N Q H L C
5′-ATCCCTGAGGAATACCTTCAG CGA TTTGTTAACCAACACTTGTGT

GG-3′

59 60 61 62 63 64 65 B1 B2 B3 B4 B5 B6 B7

ii. hir_insrev1 (SEQ ID NO: 3)

5′-CCTCACAAGTG TTGGTTAACA AA TCG CT GAAGGTATTC CTCAGGGAT-3′

iii. hirf1 (SEQ ID NO: 4, Encoded Protein Segment: SEQ ID NO: 5)

L T Y T D C
5′- TTTTTTTGGATCCTTTGGATAAAAGACTTACGTATACTGACTGCAC

-3′

(the underlined portion of this sequence is a restriction site for Kpn1)

iv. insnco1rev (SEQ ID NO: 6)

5′-TTTTTTCCAT GGGTCGACTATCAG-3′

Primer hir_insf1 described the junction between codons for the terminal amino acids of hirudin (59-65) and the insulin sequence B1-B7 via the Arg linker (codon in bold type). Primer hir_insrev1 was 100% complementary thereto. Primer hirf1 coded for the start of the hirudin gene extended to the KpnI cleavage site as described in EP-A 0 324 712, which is incorporated by reference herein in its entirety. Primer insnco1rev marked the 3′ end of the synthetic mini-proinsulin according to EP-A 0 347 781, which is incorporated by reference herein in its entirety.

Two standard polymerase chain reactions were carried out using the primer pairs hirf1/hir_insrev1 with DNA of plasmid pK152 as template and hir_insf1/insnco1rev with DNA of plasmid pSW3 as template. The reactions were carried out in 100 μl PCR buffer (as provided by the Advantage-HFTM PCR Kit (Clontech Cat' 1909-1) with, in each case, 200 nmol of each primer, 1 μl of polymerase (as provided with the kit) and 100 ng of vector. Step 1 is a 2-minute incubation at 95° C.

This was then followed by 25 cycles of 30″ at 95° C., 30″ at 55° C. and 30″ at 72° C. The last cycle was followed by an incubation at 72° C. for 3 minutes, and the reaction was subsequently stopped by cooling to 4° C. Since the primers hir_insrevkr and hir_insfkr were 100% complementary, the DNA products of the two products overlap according to said sequence so that in a third reaction under the same conditions as described above, using 5% of the PCR-fragments generated in the first two reactions as templates and the primers hirf1 and insnco1rev, a DNA fragment was formed, which encoded hirudin and mini-proinsulin separated by Arg. The PCR fragment was digested according to the manufacturer's protocol by the enzymes KpnI and NcoI and then, in a T4 ligase reaction, inserted into the pαADH2 vector opened by Kpn1/NcoI. In the same manner, except as noted below, as Example 7 of EP-A 0 347 781, which is incorporated by reference herein in its entirety, competent E. coli MM294 cells were then transformed with the ligation mixture. Plasmid DNA was then isolated from two clones for characterization by means of DNA sequence analysis. After confirmation of the inserted DNA sequence, DNA of a plasmid preparation was used to transform cells of baker's yeast strain Y79, according to said Example. However, when using the pαADH2 vector, introduction of the vector was followed by selecting for complementation of the trp1-1 mutation on yeast minimal medium agar plates, which contained no tryptophan, in contrast to said Example. For another control, plasmid DNA was reisolated from yeast transformants and analyzed by means of restriction analysis by standard techniques. The expression vector constructed was denoted pADH2Hir_Ins. Expression was carried out according to Example 4 of the present document. The fusion protein was found in the supernatant.

### EXAMPLE 2

### Construction of an Expression Cassette Encoding a Fusion Protein Made of Leu-Hirudin (Refludan®)-Gly Asn Ser Ala Arg-Mini-Proinsulin

This Example demonstrates a way of modifying the trypsin recognition site between hirudin derivative and mini-proinsulin. As discussed in more detail below, the construction was carried out similar to Example 1, except that different primers and vectors were used.

Two new oligonucleotides are synthesized:

Hir_insf (SEQ ID NO: 7, Encoded Protein Segment: SEQ ID NO: 8)

```
                                                  G N S A R F V N Q H L C
5′ ATCCCTGAGGAATACCTTCAGGGAAATTCGGCACGATTTGTTAACCAACACTTGTGTGG 3′
                                    Hir₆₅ B1 B2 P3 B4 B5 B6 B7
```

Hir_insrev (SEQ ID NO: 9)

5′ CCACACAAGTGTTGGTTAACAAATCGTGCCGAATTTCCCTGAAGGTATTCCTCAGGGAT 3′
B2 B1 Hir₆₅

(the bold letters of this sequence encode for the peptide sequence GNSAR)

Two polymerase chain reactions were carried out under the same conditions as Example 1, except using the primer pairs hirf1/hir_insrev with DNA of plasmid pK152 as template and hir_insf/insnco1rev with DNA of plasmid pSW3 as template. In a third reaction, using the products of the first two reactions as templates and the primers hirf1 and insnco1rev, a DNA fragment was formed, which encoded hirudin and mini-proinsulin separated by the linker Gly Asn Ser Ala Arg. The product of the third reaction was subsequently cleaved by KpnI and NcoI, introduced into the appropriately opened pαADH2 vector and characterized according to Example 1. The plasmid was denoted pADHH_GNSA_Ins. Cells were transformed with the plasmid DNA. Expression was carried out according to Example 3 of the present document. The fusion protein was found in the supernatant.

### EXAMPLE 3

### Expression of the Recombinant Products in the Baker's Yeast System

The expression was divided into two phases. First, a preculture was cultivated in yeast minimal medium. The culture was grown overnight in a incubation shaker at 30° C. and 240 rpm. The medium had the following composition per liter:

| 6.7 g | yeast nitrogen base (without amino acids) |
|---|---|
| 5.0 g | casamino acids (vitamin-free) |
| 0.008% | adenine |
| 0.008% | uracil |
| 2% | glucose |

As described in more detail below, the main or expression culture was inoculated with an aliquot of the preculture.

The main culture medium contained per liter:

| 10 g | yeast extract |
|---|---|
| 20 g | peptone |
| 0.008% | adenine |
| 0.008% | uracil |
| 4% | glucose |

Using the media described, expression was carried out in a shaken flask in the following way: 0.3 ml of a preculture which had been cultivated overnight was diluted with 80 ml of prewarmed medium and incubated with vigorous shaking at 30° C. for approximately 24 hours. In each case, 1 ml of the culture produced in this way was then centrifuged, after determining the optical density, and, after removing the cells, the supernatant was lyophilized and analyzed by means of SDS-PAGE. The biologically active hirudin content was determined by carrying out a thrombin inhibition assay in accordance with Example 5 of the present document. An alternative fermentation protocol, which was not conducted as part of the present Example, provides for the cells to be removed by filtration using filtration cassettes provided by Millipore or careful centrifugation at 3 to 5000×g. While isolating in parallel the protein of interest from the medium as described in Example 6, the cells were provided with fresh prewarmed main culture medium in an amount equal in volume to the original containing 1 (v/v) % ethanol and not more than 0.5% of glucose as carbon sources, and thus fermentation was continued without interruption. Although this step was only repeated once in this Example, it may be repeated up to 5 times.

### EXAMPLE 4

### Cloning and Expression of the Hirudin-Arg-Mini-Proinsulin Fusion Protein in a P. pastoris System

Invitrogen® sells a cloning and expression kit for preparing recombinant proteins with the aid of a P. pastoris system. For this, a detailed technical protocol regarding preparation and subsequent expression of the P. pastoris system for the production of a desired recombinant protein is provided so that only the construction of the expression vector encoding the desired protein has to be described when following said protocols. The EasySelect™ Pichia expression kit (catalog no. K1740-01) was used.

The pPICZαA vector was part of the kit. Opening the vector by the restriction enzymes XhoI and SacII made it possible to append, similar to Example 1 according to the manufacturer's protocol, a protein of interest to the alpha factor leader sequence and to test for secretion into the supernatant. Cloning of the fusion protein required two primers. Primer pichia_H_If1 (SEQ ID NO: 10) had the sequence:

5′ - TTTTTTTCTCGAGAAAAGA CTTACGTATACTGAC - 3′
Xhol Hir₁ Hir₂ etc.

Primer pichia_H_Irev2 (SEQ ID NO: 11) had the sequence:

5′ - TTTTTTGGCGCCGAATTCACTATTAGTTACAGTAGTTTTCC -3′
SacII EcoRI A21

The template was DNA of plasmid pADH2Hir_Ins of Example 1 of the present document. A standard PCR, under the conditions of Example 1, with both primers produced a DNA product which contained the sequence hirudin-Arg-mini-proinsulin extended by the XhoI and SacII integration sites. When the DNA product was cleaved appropriately and the fragment was isolated, said fragment was inserted into the opened vector DNA in a T4 DNA ligase reaction. In deviation from the manufacturer's protocol, E. coli strain MM294, described in Example 1, was transformed with the ligation mixture and recombinant colonies were screened for successful transformation on zeocine selection plates. Plasmid DNA was reisolated from clones and then characterized by means of restriction and DNA sequence analysis by standard techniques. Using the plasmid constructed in this way, a P. pastoris expression clone for production of the fusion protein was then prepared, following the manufacturer's instructions.

### EXAMPLE 5

### Thrombin Inhibition Assay

The hirudin concentration was determined according to the method of Grieβbach et al. (Thrombosis Research 37, pp. 347-350, 1985, which is incorporated by reference herein in its entirety). For this purpose, a Refludan® standard was included in the measurements in order to establish a calibration curve from which the yield in mg/l could be determined directly. The biological activity, as measured in accordance with the method of Grieβbach et al., was also a direct measure for correct folding of the proinsulin component of the fusion protein. Alternatively, although not performed in this Example, it is possible to use a proteolytic Staphylococcus aureus digestion and subsequent analysis in an RP-HPLC system to determine the correct S—S bridge formation.

### EXAMPLE 6

### Purification of the Fusion Protein

After completion of the fermentation of Example 4, the pH is adjusted, using concentrated H₂SO₄, to 2.5-3. In contrast to most other polypeptides found in the supernatant due to either spontaneous lysis of host cells or secretion, the fusion protein is surprisingly not precipitated at pH 2.5-3. The culture medium is therefore acidified appropriately and then, after completion of the precipitation after 30 minutes to 2 hours or longer if the scale is several m³, the precipitate and the cells are removed by centrifugation under at least 3000×g. Subsequently, the medium is adjusted, using concentrated H₂SO₄, to pH 6.8 and the fusion protein content is determined in parallel by analytical HPLC measurement. The determination is followed by adding trypsin to the supernatant so that trypsin is at approximately 1 μg per 1-1.5 mg of fusion protein. After incubation at room temperature for approximately 4 hours, purification is carried out by cation exchange chromatography using a S-hyperfine Df or Source 30S cation exchange column at pH 3.5 by concentrated H₂SO₄ in the presence of 30% (v/v) 2-propanol. Elution is carried out in the buffer by applying a linear gradient of from 0.15 to 0.45 M of NaCl. Mono-Arg-insulin is eluted at approximately 0.3 M of NaCl. After 1:1 dilution with H₂O, mono-Arg-insulin is precipitated from the insulin-containing fractions at approximately pH 6.8 with the addition of a 10% strength aqueous ZnCl₂ solution to give a final concentration of 0.1% of ZnCl₂. In this regard, the fractions are analyzed for insulin content by SDS-PAGE analysis and by Western Blot analysis. For standard Western Blot experiments the polyclonal Guinea Pig Anti-Insulin (Code NO.:A0564, DAKO Corp.) is used.

Insulin is filtered off and then dissolved in 0.05 M Tris-HCl (pH 8.5) resulting in a 2 mg/ml solution. Then, the amount of approximately 1 unit (one unit causes the hydrolysis of one micromole of hippuryl-L-arginine per minute at 25° C. and pH 7.65 under the specified conditions) of carboxypeptidase B per 100 ml solution is added and the reaction at room temperature is carried out with gentle stirring. The pH is then adjusted to pH 5.5 with citric acid, and insulin is crystallized in the presence of ZnCl₂. The crystals are removed, dissolved and, after purification by RP-HPLC, insulin is purified again by crystallization.

### EXAMPLE 7

### Processing of the Fusion Protein Directly in the Culture Medium

At the end of the expression period, the culture medium is adjusted using concentrated H₂SO₄ to pH 6.8 and trypsin is then added with stirring so that a final concentration of 4-8 mg per liter is established. After incubation at room temperature under gentle stirring for approximately 4 hours, the fermentation broth treated in this way is adjusted, using concentrated H₂SO₄, to pH 2.5-3. After 1-6 hours of precipitation, the pH is raised using NaOH to 3.5, and the mono-Arg-insulin formed is purified via cation exchange chromatography using a Source 30S chromatography column in the presence of 30% (v/v) 2-propanol. Elution is carried out by means of a linear NaCl gradient of 0.05-0.5 M salt. The product-containing fractions are diluted 1:1 with H₂O and then ZnCl₂ is added, so that a 0.1% strength ZnCl₂ solution is formed. In this regard, the fractions are analyzed for insulin by SDS-PAGE analysis and by Western Blot analysis. For standard Western Blot experiments the polyclonal Guinea Pig Anti-Insulin (Code NO.:A0564, DAKO Corp.) is used. Mono-Arg-insulin precipitates at approximately pH 6.8 and is converted to insulin according to Example 6.

While the invention has been described in connection with certain preferred embodiments so that aspects thereof may be more fully understood and appreciated, it is not intended to limit the invention to these particular embodiments. On the contrary, it is intended to cover all alternatives, modifications and equivalents as may be included within the scope of the invention as defined by the appended claims.

What is claimed is:

1. A nucleic acid sequence comprising:
Pₓ—Sₓ—Bₙ—(ZR)-Hir(AsₘR)-protein(Y)-T
where
Pₓ is a promoter sequence;
Sₓ is a nucleic acid encoding a signal sequence or leader sequence;
Bₙ is 1-15 codons, when n is an integer from 1 to 15, or a chemical bond, when n=0;
Z is a codon for lysine or arginine;
R is an arginine codon or a chemical bond;
Hir is a nucleic acid sequence coding for hirudin or hirudin derivative which is at least 40% homologous to a natural hirudin isoform;
Asₘ is a chemical bond, when m=0, or 1-10 codons, when m is an integer from 1 to 10;
protein(Y) is a nucleic acid sequence encoding a protein that is produced in and secreted by yeast; and
T is an untranslated expression-enhancing nucleic acid sequence.

2. The nucleic acid of claim 1, wherein protein(Y) encodes for mini-proinsulin or a derivative thereof.

3. The nucleic acid of claim 1, wherein protein(Y) encodes for interleukin, lymphokine, or interferon.

4. A fusion protein encoded by the nucleic acid of claim 1.

5. A fusion protein encoded by the nucleic acid of claim 2.

6. A fusion protein encoded by the nucleic acid of claim 3.

7. A multicopy vector comprising the nucleic acid of claim 1.

8. A plasmid comprising the nucleic acid of claim 1.

9. A host cell comprising the nucleic acid of claim 1, as part of the host cell chromosome, as part of a mini-chromosome, or extra-chromosomally.

10. The host cell of claim 9, wherein the host cell is a yeast.

11. The host cell of claim 10, wherein the yeast is selected from Saccharomyces cerevisiae, Kluyveromyces lactis, Hansenula polymorpha, and Pichia pastoris.

12. A host cell comprising the multicopy vector of claim 7.

13. A host cell comprising the plasmid of claim 8.

14. A process of fermentative production of fusion protein, comprising:
expressing the nucleic acid of the host cell of claim 9 to form the fusion protein in a fermentation supernatant of a cell culture; and
isolating the fusion protein from the fermentation supernatant of the cell culture.

15. The process of claim 14, wherein isolating the fusion protein comprises adjusting the pH of the fermentation supernatant to about 2.5 to 3.5 to precipitate non-desired proteins and to form a precipitation supernatant, and isolating the fusion protein from the precipitation supernatant.

16. The process of claim 15, further comprising separating the fermentation supernatant from the host cell, and after separating the fermentation supernatant from the host cell, the host cell is repeatedly cultured in fresh medium to form additional supernatant from each culture, and fusion protein is isolated from each additional supernatant.

17. The process of claim 14, wherein:
isolating the fusion protein comprises precipitating the fusion protein from the fermentation supernatant, and
the method further comprises removing the protein encoded by protein(Y) from the fusion protein, and concentrating the protein encoded by protein(Y) by at least one of microfiltration, hydrophobic interaction chromatography, and ion exchange chromatography.

18. A process of fermentative production of fusion protein, comprising:
expressing the nucleic acid of the host cell of claim 12 to form the fusion protein in a supernatant of a cell culture; and
isolating the fusion protein from the supernatant of the cell culture.

19. A process of fermentative production of fusion protein, comprising:
expressing the nucleic acid of the host cell of claim 13 to form the fusion protein in a supernatant of a cell culture; and
isolating the fusion protein from the supernatant of the cell culture.

20. A process for preparing insulin, comprising:
expressing and isolating a fusion protein by the process of claim 14;
releasing insulin into a reaction mixture by treating the fusion protein with trypsin and carboxypeptidase B; and
isolating the insulin from the reaction mixture.
